(19) United States
(12) Patent Application Publication
(10) Pub. No.: US 2013/0209378 A1
(43) Pub. Date: Aug. 15, 2013

(54) SKIN CONDITIONING AND LUBRICATING CREME, AND METHOD OF MAKING AND USING SAME

(71) Applicants: Jennifer Harris, Phoenix, AZ (US); Debra K. Butterworth, Scottsdale, AZ (US)

(72) Inventors: Jennifer Harris, Phoenix, AZ (US); Debra K. Butterworth, Scottsdale, AZ (US)

(73) Assignee: The Dial Corporation, Scottsdale, AZ (US)

(21) Appl. No.: 13/370,058

(22) Filed: Feb. 9, 2012

(51) Int. Cl. A61K 8/58; A61Q 19/08; A61Q 19/00; A61K 8/92; A61Q 17/04; A61K 8/97; A61K 8/98; A61K 8/67; A61Q 9/04

(52) U.S. Cl. 424/59; 424/683

(57) ABSTRACT

A cosmetic agent that takes the place of powdered talc is described herein. The present cosmetic agent is a skin conditioning and lubricating crème comprising talc; at least one volatile silicone fluid; at least one structuring component; and at least one emollient. The inventive composition provides skin conditioning and lubrication without the inconveniences inherent in using powdered products such as talcum powder.

### FIELD OF THE INVENTION

The present invention generally relates to cosmetic agents and more particularly to a skin conditioning and lubricating crème comprising talc, method of making such a crème, and method for conditioning and lubricating skin with such a crème.

### BACKGROUND OF THE INVENTION

Talc is hydrated magnesium silicate, corresponding to the chemical formula Mg₃Si₄O₁₀(OH)₂. It is an exceptionally well known cosmetic agent that has been used for decades, perhaps centuries, in the powdered form known as talcum powder. The most common use for talcum powder is the treatment and prevention of skin rashes, such as diaper rash, and for general personal grooming. It is a remarkable dry lubricant, imparting a slick feel to skin due to the slipping platelet crystal structure.

Some of the more recent work with talc was to improve transparency, skin adhesiveness, lubricity, spreadability, moisture absorbency, oil retention, fragrance retention, and other properties. In line with the objective to improve these properties of talc itself, U.S. Pat. No. 5,849,333 (Nordhauser et al.) and U.S. Pat. No. 6,132,744 (Chehab et al.) give excellent backgrounds in talc crystal structure and teach modifications to talc to improve some of these above-mentioned properties.

Besides powdered cosmetic agents consisting of talc itself, or perhaps talc with other dry powders such as corn starch, talc has been formulated into countless cosmetic agents such as bar soap, makeup, and antiperspirants. Some of the more relevant and interesting cosmetic compositions comprising talc are discussed below.

U.S. Pat. No. 4,800,076 (Bhat et al.) discloses skin care compositions having improved aesthetic and physical characteristics comprising a lotion, cream, or anhydrous base and talc, wherein the ratio of non-volatile ingredients to talc is from 0.4:1 up to 3.2:1. The exemplary embodiments in the disclosure appear to be complex compositions with a large number of ingredients. Interestingly, the disclosure recites that, “[t]he essence of the present invention is not within the composition of the base per se and any of the many formulations or compositions of the lotion, cream or anhydrous type currently utilized in skin care preparations can be employed,” (Col 2, Lines 28-32).

U.S. Pat. No. 5,338,535 (Berndt) discloses a non-aqueous “liquid powder,” defined as a lotion or cream that dries into a powder on skin after drying. The Berndt product is claimed to comprise 25 to 75% of a starch powder and 25 to 75% of a volatile cyclomethicone. There are no claims to talc being the left-behind powder after drying on the skin.

U.S. Pat. No. 5,882,657 (Miguel-Colombel et al.) discloses a cosmetic composition in the form of a soft paste that comprises wax, oil, a thickener and filler. The filler is chosen to absorb at least some of the oil in the composition and is chosen from talc, mica, silica, kaolin, nylon powder, polyethylene powder, and the like.

U.S. Pat. Nos. 6,120,781 and 6,132,742 (Le Bras et al.) discloses soft paste cosmetic compositions comprising in a fatty phase from 12-60% by weight of a wax with melting point above 55° C.

U.S. Application Publication No. 2002/0001572 (Brewster et al.) discloses a soft-solid antiperspirant comprising (a) a volatile silicone hydrocarbon; (b) a structuring wax; (c) a silicone elastomer; and (d) an antiperspirant or deodorant active. Optional ingredients for the soft-solid products include drying agents, disclosed to include such substances as talc or aluminum starch octenylsuccinate.

U.S. Application Publication No. 2004/0197286 (Robert et al.) discloses a makeup composition comprising at least one fatty acid ester or fatty alcohol ester and at least one silicone compound. The inventive cosmetic composition may also comprise filler, disclosed to include talc.

U.S. Application Publication No. 2005/0058669 (Krzysik et al.) discloses a topical ointment for skin that comprises an emollient, a structurant, a rheology enhancer, and other optional ingredients. The inventive ointments may also comprise a particulate material, disclosed to include talc.

U.S. Pat. No. 6,949,249 (Healy et al.) discloses a protective spray for skin comprising one or more active agents, one or more rheology modifiers, and a carrier. In this invention, talc is discloses as one of the actives for protecting skin and thus may be included in the compositions.

Lastly, U.S. Application Publication No. 2006/0159645 discloses a skin lubricant comprising a blend of silicone and particulate. In this case, the particulate is selected from the group of silica, talc, and cornstarch.

In spite of the developments in cosmetic agents comprising talc, no actual skin conditioners and skin lubricants are known that can compete with talcum powder for convenience and cost. Therefore, one object of the present invention is to discover new cosmetic agents for conditioning and lubricating skin that are easy and pleasurable to use with a luxurious texture, which are relatively inexpensive and not overly burdensome to manufacture.

### BRIEF SUMMARY OF THE INVENTION

The present invention is a skin conditioning and lubricating crème comprising talc; at least one volatile silicone fluid; at least one structurant; and at least one emollient. The present invention also comprises a method for producing the crème and a method for conditioning and/or lubricating skin with said crème.

In a preferred embodiment of the present invention, a skin conditioning and lubricating crème comprising a blend of talc, a volatile silicone fluid, at least two structuring substances, and at least two emollients is provided in the form of a smooth thick cream having a penetration force value of from 100 g to about 200 g measured on a texture analyzer at 21° C. and 50% relative humidity using a standardized 30° conical probe pulled through the product for a distance of 5 mm at a rate of 1 mm per second.

In another exemplary embodiment of the present invention, a skin conditioning and lubricating crème comprising talc, volatile silicone fluid(s), structurant(s) and emollient(s) is made by a manufacturing process comprising the steps of: (a) charging a mixer with said talc and silicone fluid(s) and homogenizing until a dispersed premix forms; (b) heating, melting, and mixing in a separate heated mixing tank the structurant(s) and emollient(s); (c) adding the talc/fluid(s) premix to the molten structurant(s)/emollient(s) mixture of (b) and mixing until homogeneous; and (d) adding any fragrance or adjuvant.

In another exemplary embodiment of the present invention, skin crème comprising talc, at least one volatile silicone fluid, at least one structurant, at least one emollient, optional fragrance, and optional adjuvant is applied to, spread, and/or rubbed, onto external surfaces of the human body to provide a skin conditioning and/or lubricating effect.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention. Various changes to the described embodiments may be made, for example in the function and relative amounts of the ingredients described without departing from the scope of the invention as set forth in the appended claims.

That being said, the present invention is a skin conditioning and lubricating crème that may be used in place of talcum powder. The crème herein minimally comprises: talc; at least one volatile silicone fluid; at least one structuring component; and at least one emollient. The crème may also comprise fragrance, colorants, and/or other adjuvant to make the product more shelf stable, aesthetically acceptable, cosmetically acceptable, commercially successful, and consumer acceptable.

### Talc

As used herein, the term “talc” denotes a mineral consisting entirely or almost entirely of hydrated magnesium silicate. Talc may generally be described by either of the following formulas: H₂Mg₃(SiO₃)₄ or Mg₃Si₄O₁₀(OH)₂, with theoretically 31.7% MgO, 63% SiO₂, and 4.8% H₂O by weight. Other very minor elements and impurities may be included in the general talc crystal structure as well. Preferably, talc in the form of talcum powder is utilized. Talc is commonly preferred as filler in cosmetic agents due, at least in part, to its lubricating effects and to its ability to increase the overall moisture content of compositions. Here, the talc is important for the resulting skin feel and lubricity on the skin after application of the inventive cosmetic crème with talc.

Talc has a primarily monoclinic crystal lattice system as shown in scanning electron microscope (SEM) imaging. In crystallography, the monoclinic lattice system is one of the seven lattice point groups. A crystal system is described by three vectors. In the monoclinic system the crystal is described by vectors of unequal length. They form a rectangular prism with a parallelogram as its base. Thus, two pairs of vectors are perpendicular, while the third pair makes an angle other than 90°. In contrast, in a triclinic crystal system is another of the seven lattice point groups and is also described by three basis vectors in which the crystal has vectors of unequal length, but in the triclinic system all three vectors are not mutually orthogonal. It is the only lattice type that has no mirror planes.

That being said, the preferred talc for use in the present invention is any form of talc such as monoclinic, triclinic, or a mixture of crystal structures, and any particle size. The talc is preferably incorporated in the present cosmetic crème at a level of from about 1 wt. % to about 15 wt. %, based on the total weight of the composition. More preferred is to incorporate the talc at from about 5 wt. % to about 10 wt. %, based on the total weight of the composition.

### Volatile Silicone Component

The crème of the present invention comprises one or more volatile silicone fluids in any combination. These materials include the cyclic polydimethylsiloxanes, also known as cyclomethicones, which preferably have from about 3 to about 6 silicon atoms, and the linear polydimethylsiloxanes, also known as dimethicones, which preferably have from about 2 to about 9 silicon atoms. The cyclomethicones normally exhibit viscosities below 10⁻⁵ m²/sec (10 centistokes) and above 10⁻⁷ m²/sec (0.1 centistokes), and the dimethicones normally exhibit a viscosity below 5×10⁻⁶ m²/sec (5 centistokes). Cyclomethicones may include the silicone fluids DC® 244, DC® 245, DC® 246, DC® 344, and DC® 345, all of which are available from Dow Corning Corporation, Silicone 7207® and Silicone 7158® from Union Carbide Corporation; and SF1202® from General Electric. In at least one preferred embodiment of the cosmetic crème of the present invention, the volatile silicone fluid comprises decamethylcyclopentasiloxane, available as DC® 245 from Dow. For purposes of this invention, the volatile silicone fluid may be present in the cosmetic crème composition in an amount of from 20 to 70 wt. % and most preferably at about 40 to 60 wt. %. In at least one embodiment of particular interest, the volatile silicone is present in the cosmetic crème at a level of about 50 wt. %.

Non-volatile silicones include such materials as polyalkylsiloxanes, polyalkylaryl siloxanes, and polyether siloxanes with viscosities of about 5 to about 100,000 centistokes at 25° C., polymethylphenylsiloxanes with viscosities of about 15 to about 65 centistokes, and polyoxyalkylene ether dimethylsiloxane copolymers with viscosities of about 1200 to about 1500 centistokes. These materials may also function more as emollients, and may be included in the cosmetic crème compositions as such (detailed below). If included, the non-volatile silicones may be present as emollients from about 5 wt. % to about 25 wt. %.

### Structuring Component

The cosmetic crème of the present invention necessarily includes as least one structuring component, referred to in the cosmetic arts as a “structurant.” The term “structurant” as used herein refers to any cosmetically acceptable material known or otherwise effective in providing suspending, gelling, viscosifying, solidifying, and/or thickening to a cosmetic composition or to otherwise provide physical structure to the final product form. The preferred structurants for use herein include waxes, fats, fatty acid salts, fatty alcohols, hydrogenated oils, gellants, polymers, cellulosic materials, various sorbitols, and combinations thereof. Such materials will typically be solids under ambient conditions and will be melted either before addition to a batch mixture or at least heated beyond their melting points while in the mixing tank.

Some of the more suitable structurants for use herein include cellulosic thickeners such as hydroxy propyl cellulose and hydroxy ethyl cellulose, sodium stearate, hydroxystearic acid, dibutyl lauroyl glutamide, alkyl silicone waxes, quaternium-18 bentonite, quaternium-18 hectorite, propylene carbonate, hydrogenated castor or jojoba oil (e.g., Castorwax); natural animal or plant oils/waxes and semi-synthetic oils/waxes such as waxes derived from avocado, linseed, almonds, Ibota wax, perilla oil, olive oil, cacao butter, kapok wax, kaya oil, carnauba wax, glycyrrhiza oil, candelilla wax, beef tallow, neat's-foot oil, beef bone fat, hydrogenated beef tallow, apricot kernel oil, spermaceti wax, hydrogenated oils such as wheat germ oil, sesame oil, rice germ oil, rice bran oil, sugar cane wax, sasanqua oil, safflower oil, shear butter, Chinese tung oil, cinnamon oil, jojoba wax, shellac wax, turtle oil, soybean oil, tea seed oil, camellia oil, evening primrose oil, corn oil, lard, rapeseed oil, Japanese tung oil, rice bran oil, germ oil, horse fat, persic oil, palm oil, palm kernel oil, castor oil, castor oil fatty acid methylester, sunflower oil, grape oil, bayberry wax, jojoba oil, macadamia nut oil, beeswax, mink oil, cottonseed oil, cotton wax, Japanese wax, Japanese wax kernel oil, montan wax, coconut oil, hydrogenated coconut oil, tri-coconut oil fatty acid glyceride, mutton tallow, peanut oil, lanolin, liquid lanolin, hydrogenated lanolin, lanolin alcohol, hard lanolin, lanolin acetate, isopropyl lanolate, hexyl laurate, poly(oxyethylene) lanolin alcohol ether, poly(oxyethylene) lanolin alcohol acetate, polyethylene glycol lanolate, poly(oxyethylene) hydrogenated lanolin alcohol ether, egg yolk oil, the synthetic waxes such as Fisher-Tropsch waxes and microcrystalline wax; polyethylene with molecular weight of 200 to 1000 daltons, polyethylene-vinyl acetate copolymers, substituted and unsubstituted dibenzylidene alditols (e.g. dibenzylidene sorbitol); and combinations thereof.

Polyethylene with molecular weight from about 200 to about 1,000 Daltons is especially preferred. These materials are available as Performalene® 400, Performalene® 500, and Performalene® 655 from New Phase Technologies.

Fatty alcohols are especially preferred as structurants in the present compositions. Some of the more preferred fatty alcohol structurants include the linear or branched, saturated or unsaturated fatty alcohols having a total of 10-20 carbon atoms (i.e. inclusive of C₁₀₋₂₀ fatty alcohols, whether saturated or unsaturated, branched or linear). Fatty alcohols for use herein include, but are not limited to, capric alcohol (1-decanol, decyl alcohol, C₁₀H₂₂O), lauryl alcohol (dodecanol, 1-dodecanol, C₁₂H₂₆O), myristyl alcohol (1-tetradecanol, C₁₄H₃₀O), cetyl alcohol (1-hexadecanol, C₁₆H₃₄O), palmitoleyl alcohol (cis-9-hexadecen-1-ol, C₁₆H₃₂O), stearyl alcohol (1-octadecanol, C₁₈H₃₈O), isostearyl alcohol (16-methylheptadecan-1-ol, branched C₁₈H₃₈O), oleyl alcohol (9-cis-octadecen-1-ol, C₁₈H₃₆O), elaidyl alcohol (9-trans-octadecen-1-ol, C₁₈H₃₆O), linoleyl alcohol (all-cis-9,12-octadecadien-1-ol, C₁₈H₃₄O), linolenyl alcohol (all-cis-9,12,15-octadecatrien-1-ol, C₁₈H₃₂O), arachidyl alcohol (1-eicosanol, C₂₀H₄₂O), gadoleyl alcohol (9-cis-eicosen-1-ol, C₂₀H₄₀O), 5,8,11,14-eicosatetraen-1-ol (C₂₀H₃₄O), and any combination thereof. The more preferred fatty alcohols for use in the present cosmetic crème include the following C₁₄-C₁₈ alcohols: myristyl alcohol (1-tetradecanol, C₁₄H₃₀O), cetyl alcohol (1-hexadecanol, C₁₆H₃₄O), palmitoleyl alcohol (cis-9-hexadecen-1-ol, C₁₆H₃₂O), stearyl alcohol (1-octadecanol, C₁₈H₃₈O), isostearyl alcohol (16-methylheptadecan-1-ol, branched C₁₈H₃₈O), oleyl alcohol (9-cis-octadecen-1-ol, C₁₈H₃₆O), elaidyl alcohol (9-trans-octadecen-1-ol, C₁₈H₃₆O), linoleyl alcohol (all-cis-9,12-octadecadien-1-ol, C₁₈H₃₄O), and linolenyl alcohol (all-cis-9,12,15-octadecatrien-1-ol, C₁₈H₃₂O). The most preferred fatty alcohols include myristyl alcohol (1-tetradecanol, C₁₄H₃₀O), cetyl alcohol (1-hexadecanol, C₁₆H₃₄O), and stearyl alcohol (1-octadecanol, C₁₈H₃₈O), and mixtures thereof. Cetearyl alcohol is a commercially available mixture of cetyl alcohol and stearyl alcohol (e.g. Lipocol® CS-50 or CS-604 from Lipo Chemicals Inc.) and is also preferred for use herein.

The at least one structurant is incorporated into the cosmetic crème composition at from about 15 wt. % to about 35 wt. %, and more preferably at from about 20 wt. % to about 30 wt. %, based on the total weight of the cosmetic crème composition. Most preferred is to incorporate a mixture of structurants comprising any of the following substances at from about 20 to about 30 wt. %, based on the total weight of the crème composition: stearyl alcohol, cetyl alcohol, cetearyl alcohol, myristyl alcohol, hydrogenated castor oil, hydrogenated Jojoba oil, lanolin, paraffin wax, beeswax, carnauba wax, candelilla wax, spermaceti wax, ozokerite (ozocerite) wax, ceresin wax, montan wax, polyethylene of molecular weight from 200 to 1000 daltons, and mixtures thereof.

### Emollient

The crème in accordance with the present invention necessarily includes one or more emollients. These emollients are typically considered “non-volatile.” One or more emollients are incorporated in the cosmetic crème composition of the present invention to impart consumer acceptable feel to the crème itself and to render emollient effects to the skin to which the product is applied.

Suitable non-volatile emollients include silicone and non-silicone materials. Such silicone materials include polyalkyl siloxanes, polyalkyaryl siloxanes, and polyether siloxane copolymers. Non-silicone materials may include fatty acids, fatty alcohol esters (e.g. “ester oils”), and water insoluble ethers and alcohols. Emollients typically used in personal care compositions are described in Cosmetic, Science and Technology, Vol, 1, 27-104 (1972, Balsam and Sagarin editors), incorporated herein by reference in its entirety. Emollients may be incorporated in the crème composition from about 5% to about 25% by weight of the total composition.

Other suitable emollients include the polyethylene and/or polypropylene glycol ethers of C₄₋₂₀ alcohols, including such materials as PPG-10 Butanediol, PPG-14 Butyl Ether, PPG-5-Buteth-7, PPG-3-Isostearth-9, PPG-3-Myreth-3, Oleth-10, and Steareth-20. Polyethylene and/or polypropylene glycol esters of fatty acids include PEG-8 Distearate, PEG-10 Dioleate, and PPG-26 Oleate.

Other preferred emollients for use in the present invention include the ester oils such as diisobutyl adipate, 2-hexyldecyl adipate, di-2-heptylundecyladipate, N-alkyl glycolmonoisostearate, isocetyl isostearate, trimethylolpropane triisostearate, ethylene glycol di-2-ethylhexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, isononyl isononanate, neopentyl glycol dicaprirate, triethyl citrate, 2-ethylhexyl succinate, amyl acetate, ethyl acetate, butyl acetate, isocetyl stearate, butyl stearate, diisopropyl sebacinate, di-2-ethylhexyl sebacinate, cetyl lactate, myristyl lactate, isopropyl palmitate, 2-ethylhexyl palmitate, 2-hexyldecyl palmitate, 2-heptylundecyl palmitate, cholesteryl 12-hydroxystearate, dipentaerythritol fatty acid esters, isopropyl myristate, octyldodecyl myristate, 2-hexyldecyl myristate, myristyl myristate, hexyldecyl dimethyloctanoate, ethyl laurate, hexyl laurate, 2-octyldodecyl N-lauroyl-L-glutamate, and diisostearyl malate; and glyceride oils, e.g., acetoglyceryl, glycerol triisooctanoate, glyceryl triisostearate, glyceryl triisopalmitate, glyceryl monostearate, glyceryl di-2-heptylundecanoate, glyceryl trimyristate, diglyceryl myristyl isostearate, and mixtures thereof.

Most preferred for the crème composition is to include from about 5 to about 25 wt. % of a mixture of emollients chosen from polyethylene glycol ethers of C₄₋₂₀ alcohols, polypropylene glycol ethers of C₄₋₂₀ alcohols, non-volatile silicones, ester oils, and mixtures thereof. Of particular use is a combination of PPG-14 butyl ether and myristyl myristate at from about 5 to about 25 wt. %, a more preferably from about 10 to about 20 wt. %, based on the total weight of the crème composition.

### Solvent Component (Optional)

The crème composition is preferably anhydrous. However, it may include at least one non-water solvent as a way to adjust final viscosity and feel of the product, and to influence the evaporation of volatiles on the skin after application. Most preferred are the hydric solvents such as alcohols, glycols, and polyols. Preferred for use herein include materials such as glycerin, sorbitol, propylene glycol, dipropylene glycol, 1,3-butylene glycol, pentylene glycol, hexylene glycol, glucose, xylitol, malitol, polyethylene glycol or polypropylene glycol (typically having molecular weights of about 500 to 6000, such as PEG-10, PEG-40, PEG-150 and PPG-20), pyrrolidone carboxylate, polyoxyethylene glycoside, polyoxypropylene methylglycoside, and any combinations thereof.

If any solvent is included in the crème compositions of the present invention, the preferred solvent level is from about 0.001 wt. % to about 2 wt. %.

### Dyes, Fragrances, Preservatives, Etc. (Optional)

The cosmetic crème compositions of the present invention may also include fragrances to make the product more pleasant for the user. Fragrances for use in personal care compositions are generally available from the fragrance supply houses such as International Flavors & Fragrances (IFF), Symrise, Givaudan, Firmenich, Robertet, H & R, amongst others, and may be added to the crème composition to provide both odor masking, complexing of odorous materials through fragrance accords, or general “perfuming” for the product itself and to impart substantive fragrance to the skin. Fragrances may be added to the compositions at supplier recommended levels, or from about 0.001 to about 5 wt. %.

The fragrances for use herein may include a broader class of natural products comprising natural oils extracted from plants and trees and their fruits, nuts and seeds, (for example by steam or liquid extraction of ground-up plant/tree material), natural products that may be purified by distillation, (i.e., purified single organic molecules or close boiling point “cuts” of organic materials such as terpenes and the like), and synthetic organic materials that are the synthetic versions of natural materials (e.g., either identical to the natural material or perhaps the optical isomer, or the racemic mixture). An example of the latter is d,l-limonene that is synthetically prepared and is a good and eco-friendly substitute for natural orange oil (mostly d-limonene) when crop yields are expensive due to citrus crop freezes. It is understood that “fragrances” usually incorporate a wide range of pure organic materials either natural or synthetic, mixtures of these previously purified individual materials or distillate cuts of materials, and complex natural mixtures directly extracted from plant/tree materials through infusion, steam extraction, etc., and in fact many natural extracts, oils, essences, infusions and such are very fragrant materials.

Some of the naturally derived materials that may be of use in the present compositions include, but are not limited to, musk, civet, ambergis, castoreum and similar animal derived oils; abies oil, ajowan oil, almond oil, ambrette seed absolute, angelic root oil, anise oil, basil oil, bay oil, benzoin resinoid, bergamot oil, birch oil, bois de rose oil, broom abs., cajeput oil, cananga oil, capsicum oil, caraway oil, cardamon oil, carrot seed oil, cassia oil, cedar leaf oil, cedar wood oil, celery seed oil, cinnamon bark oil, citronella oil, clary sage oil, clove oil, cognac oil, coriander oil, cubeb oil, cumin oil, camphor oil, dill oil, elemi gum, estragon oil, eucalyptol nat., eucalyptus oil, fennel sweet oil, galbanum res., garlic oil, geranium oil, ginger oil, grapefruit oil, hop oil, hyacinth abs., jasmin abs., juniper berry oil, labdanum res., lavender oil, laurel leaf oil, lavender oil, lemon oil, lemongrass oil, lime oil, lovage oil, mace oil, mandarin oil, mimosa abs., myrrh abs., mustard oil, narcissus abs., neroli bigarade oil, nutmeg oil, oakmoss abs., olibanum res., onion oil, opoponax res., orange oil, orange flower oil, origanum, orris concrete, pepper oil, peppermint oil, peru balsam, petitgrain oil, pine needle oil, rose abs., rose oil, rosemary oil, safe officinalis oil, sandalwood oil, sage oil, spearmint oil, styrax oil, thyme oil, tolu balsam, tonka beans abs., tuberose abs., turpentine oil, vanilla beans abs., vetiver oil, violet leaf abs., ylang ylang oil and similar vegetable oils, etc.

Synthetic essences include but are not limited to pinene, limonene and like hydrocarbons; 3,3,5-trimethylcyclohexanol, linalool, geraniol, nerol, citronellol, menthol, borneol, borneyl methoxy cyclohexanol, benzyl alcohol, anise alcohol, cinnamyl alcohol, β-phenyl ethyl alcohol, cis-3-hexenol, terpineol and like alcohols; anethole, musk xylol, isoeugenol, methyl eugenol and like phenols; α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hyxylcinnamic aldehyde, undecenal, heliotropin, vanillin, ethyl vanillin and like aldehydes; methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone and like ketones; amyl butyrolactone, diphenyl oxide, methyl phenyl glycidate, gamma.-nonyl lactone, coumarin, cineole, ethyl methyl phenyl glicydate and like lactones or oxides; methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, vetiveryl acetate and the like.

Encapsulated fragrances are well known in the art, and may find use in the crème compositions of the present invention to give the product a longer-lasting fragrance impression (better retention of the fragrance in storage) along with extended fragrance release on the skin (through mechanical or moisture/sweat rupture of the fragrance caps). Encapsulation of fragrance has been described in many prior art references, including but not limited to; U.S. Pat. No. 7,338,928 to Lau et al.; U.S. Pat. No. 7,294,612 to Popplewell et al.; U.S. Pat. No. 7,196,049 to Brain et al.; U.S. Pat. No. 7,125,835 to Bennett et al.; U.S. Pat. No. 7,122,512 to Brain et al.; U.S. Pat. No. 7,119,057 to Popplewell et al.; U.S. Pat. No. 6,147,046 to Shefer et al.; U.S. Pat. No. 6,142,398 to Shefer et al.; U.S. Pat. No. 4,446,032 to Munteanu et al.; and, U.S. Pat. No. 4,464,271 to Munteanu, each of which is incorporated herein by reference. Fragrance encapsulation has been optimized in the trade and is readily available through various suppliers, most notably LIPO Technologies, Inc., Vandalia, Ohio, and Alco Chemical, Chattanooga, Tenn., (e.g. using Alcocap® natural polymers for encapsulation). Encapsulation is described thoroughly in “Microencapsulation: Methods and Industrial Applications”, Benita (Ed.), Marcel Dekker, Inc., New York, 1996. Fragrance microcapsules obtained from LIPO, Alco, or the fragrance houses, or as obtained through any of these published methods may be incorporated in the crème compositions herein at from about 0.001% to about 5% by weight of the total composition.

### Optional Adjuvant

Cosmetic skin crème compositions in accordance with the present invention may also include dyes, pigments, UV filters/blockers, depilatory agents, preservatives, antioxidants, skin pigment modifiers (e.g. instant tan technology), vitamins, conditioners, anti-wrinkle agents, and the like. Dyes may comprise pigments, or other colorants, chosen so that they are compatible with the other ingredients in the crème composition, compatible with the manufacturing process, and not staining to skin or clothing. For example, a preferred colorant for use in the present invention is at from about 0.0001% to about 0.1% by weight of the total composition. Non-limiting examples of dyes include Blue 1 (CI 420980), Yellow 8 (CI 43350), Liquitint® Green FS (from Milliken), C.I. Pigment Green #7, C.I. Reactive Green #12, F D & C Green #3, C.I. Acid Blue #80, C.I. Acid Yellow #17, Liquitint® Red MX, F D & C Yellow #5, Liquitint® Violet LS, Fast Turquise GLL, Liquitint® Blue MC, and Liquitint® Blue HP, and mixtures thereof.

Examples of suitable UV filters include 4-aminobenzoic acid; 3-(4′-trimethylammonium) benzylideneboran-2-one methylsulfate; 3,3,5-trimethylcyclohexylsalicylate; 2-hydroxy-4-methoxybenzophenone; 2-phenyl benzimidazole-5-sulfonic acid and its potassium, sodium, and triethanolamine salts; 3,3′-(1,4-phenylenedimethine)bis-(7,7-dimethyl-2-oxobicyclo[2.2.1]heptane-1-methane sulfonic acid and its salts; 1-(4-tert-butylphenyl)-3-(4-methoxyphenyl)propane-1,3-dione, 3-(4′-sulfo)benzylidene-bornan-2-one and its salts; 2-ethylhexyl 2-cyano-3,3-diphenylacrylate; polymer of N-[2(and 4)-(2-oxoborn-3-ylidenemethyl)benzyl]acrylamide; 2-ethylhexyl 4-methoxycinnamate; ethoxylated ethyl 4-aminobenzoate; isoamyl 4-methoxycinnamate; 2,4,6-tris[p-(2-ethylhexyloxycarbonyl)anilino]-1,3,5-triazine; 2-(2H-benzotriazol-2-yl)4-methyl-6-(2-methyl-3-(1,3,3,3-tetramethyl-1-(trimethylsilyloxy) silyloxy) disiloxanyl)propyl)phenol; 4,4′-[(6-[4-((1,1-dimethylethyl)aminocarbonyl)-phenylamino]-1,3,5 -triazin-2,4-yl) diimino]bis(benzacid 2-ethylhexyl ester); 3-(4′-methylbenzylidene) D,L-camphor; 3-benzylidene camphor; 2-ethylhexylsalicylate; 2-ethylhexyl-4-dimethylaminobenzoate; hydroxy-4-methoxybenzophenone phenone-5-sulfonic acid (sulisobenzone) and the sodium salt; and, 4-isopropylbenzyl salicylate, and mixtures thereof.

Conventional preservatives may be added to the crème compositions to improve shelf life by inhibiting mold and bacteria growth and/or preventing oxidation. These preservatives typically include BHT, benzoic acid, benzoate salts, sorbic acid, sorbate salts, citric acid, citrate salts, ascorbic acid, ascorbate salts, and the parabens (esters of p-hydroxybenzoic acid). These materials are added at supplier recommended levels or at the level necessary to preserve the product for a desired shelf life.

### Exemplary Composition

TABLE 1 delineates an exemplary cosmetic crème composition (Formula 1) in accordance with the present invention that has a beautiful feel in both the hands and when rubbed onto skin.

TABLE 1
Skin conditioning and lubricating crème

| Ingredients (weight percent actives) | Formula 1 |
|---|---|
| Cyclopentasiloxane | 51.5 |
| Stearyl alcohol | 18.0 |
| Cetyl alcohol | 2.5 |
| Hydrogenated castor oil | 3.0 |
| Polyethylene | 1.1 |
| PPG-14 butyl ether | 14.0 |
| Myristyl myristate | 1.9 |
| Fragrance | 1.0 |
| Talc | 7.0 |
| Total | 100.0 |

Formula 1 from TABLE 1 exhibited the desired rheology necessary for a cosmetic crème intended to be rubbed into the skin by a consumer. The crèmes of the present invention have a penetration force value of from about 100 g to about 200 g, and more preferably from about 110 g to 160 g at 21° C. and 50% relative humidity, (as measured with a Stable Micro Systems Texture Analyzer from Texture Technologies Corp.). This value denotes the force required to move a standardized 30° conical probe through the product for a distance of 5 mm at a rate of 1 mm per second.

The present invention also comprises a method of manufacturing a skin conditioning and lubricating crème comprising: talc; at least one volatile silicone fluid; at least one structurant; at least one emollient; and optional solvent, fragrance and adjuvant. The method comprises the steps of: (a) providing a mixing vessel equipped for homogenization and charging said vessel with the at least one volatile silicone fluid and talc, homogenizing until a completely dispersed premix is formed; (b) providing a mixing tank equipped for heating, combining the at least one structurant and at least one emollient, heating beyond their melting points and mixing until smooth; then (c) adding the premix to the stirred molten mixture of structurant(s) and emollient(s).

Some nuances exist for this generalized procedure disclosed above. The final appearance and structure of the crème depends on the degree of homogenization of the talc/silicone fluid premix. This homogenization is normally conducted at room temperature and for as long as necessary to produce a premix that appears to comprise fully dispersed talc without visible lumps. The heated mixture of structurant(s) and emollient(s) is normally heated to less than about 85° C., but at least to the degree necessary to melt the waxy ingredients and allow for mixing As mentioned above, many of the structurants and emollients are waxes at room temperature and require heating to form a co-melt. This co-melt is stirred, slowly added agitation as the ingredients thoroughly melt, keeping the temperature at no greater than about 85° C. After the waxy structurant(s) and emollient(s) are co-melted and thoroughly mixed, the talc/silicone fluid premix is added to the molten structurant(s)/emollient(s) mixture, with the batch maintained at around 60° C. This mixture is stirred until homogeneous, after which time any other ingredients may be added. These additional ingredients include such substances as fragrance, dyes, colorants, preservatives, antioxidants, and the like. These remaining ingredients are added in ways that are common and customary to the cosmetic formulating industry. For example, dyes, colorants and/or preservatives may be premixed in a suitable solvent and the resulting liquid mixture added to the batch. Fragrance oils may be added directly without prior dilution. Once the crème composition is complete, the batch may be pumped out to filling lines or any other manufacturing process necessary to dispense the completed cosmetic composition into containers. The nature of the containers is beyond the scope of the present invention, but is reasoned to be some sort of point-of-purchase unit such as cosmetic tubes or jars. The containerized product is the preferred locale for the crème to cool and stiffen.

The present invention also comprises a method of conditioning and/or lubricating skin with a crème comprising at least one volatile silicone fluid, at least one structurant, at least one emollient, talc, optional solvent, optional fragrance and optional adjuvant. The method of use comprises the steps of: (a) providing cosmetic crème comprising talc, at least one volatile silicone fluid, at least one structurant, and at least one emollient; and (2) rubbing a sufficient amount of said crème into the skin to provide a conditioning and/or lubricating benefit. The crème in accordance with the present invention provides incredible conditioning of the skin on the forearms, elbows, feet and knees of persons. In many instances the crème of the present invention will replace powdered skin products such as talcum powder.

We have thus described a cosmetic crème that is a dispersed and thickened form of talc, which provides all the conditioning and lubricating effects of talc in a pleasing and convenient crème form rather than in a powdered form.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

We claim:

1. A skin conditioning and lubricating crème comprising:
a. from about 20 to about 70 wt. % of at least one volatile silicone fluid;
b. from about 15 to about 35 wt. % of at least one structurant;
c. from about 5 to about 25 wt. % of at least one emollient; and
d. from about 1 to about 15 wt. % talc,
wherein said crème has a final penetration force value of from 100 g to about 200 g measured on a texture analyzer at 21° C. and 50% relative humidity with a standardized 30° conical probe pulled through the product for a distance of 5 mm at a rate of 1 mm per second.

2. The crème of claim 1, wherein said at least one volatile silicone is decamethylcyclopentasiloxane.

3. The crème of claim 1, wherein said structurant is selected from the group consisting of stearyl alcohol, cetyl alcohol, cetearyl alcohol, myristyl alcohol, hydrogenated castor oil, hydrogenated Jojoba oil, lanolin, paraffin wax, beeswax, carnauba wax, candelilla wax, spermaceti wax, polyethylene of molecular weight from 200 to 1000 daltons, and mixtures thereof.

4. The crème of claim 1, wherein said emollient is selected from the group consisting of polyethylene glycol ethers of C₄₋₂₀ alcohols, polypropylene glycol ethers of C₄₋₂₀ alcohols, non-volatile silicones, ester oils, and mixtures thereof.

5. The crème of claim 4, wherein said emollient is selected from the group consisting of polypropylene glycol ethers of C₄₋₂₀ alcohols, ester oils, and mixtures thereof.

6. The crème of claim 1 further comprising a fragrance.

7. The crème of claim 1 further comprising adjuvant selected from the group consisting of dyes, pigments, UV filters/blockers, depilatory agents, preservatives, antioxidants, skin pigment modifiers, vitamins, conditioners, anti-wrinkle agents, and mixtures thereof.

8. A method for manufacturing the crème of claim 1, said method comprising the steps of:
a. homogenizing a mixture of said talc in said at least one volatile silicone fluid to form a talc/silicone fluid premix;
b. mixing and co-melting said at least one structurant and said at least one emollient to form a heated co-melt; and
c. blending said premix into said co-melt with stirring until homogeneous.

9. A method for the conditioning and lubrication of skin, comprising the steps of:
a. providing the crème of claim 1; and
b. rubbing a sufficient amount of said crème into the skin to provide a conditioning and/or lubricating benefit to skin.
